(19) United States
(12) Patent Application Publication
(10) Pub. No.: US 2010/0286476 A1
(43) Pub. Date: Nov. 11, 2010

(54) Endoscope With Tunable-Focus Microlens

(76) Inventors: Hongrui Jiang, Madison, WI (US); Xuefeng Zeng, Madison, WI (US)

Correspondence Address: BOYLE FREDRICKSON S.C., 840 North Plankinton Avenue, MILWAUKEE, WI, 53203, US

(21) Appl. No.: 12/435,829

(22) Filed: May 5, 2009

(51) Int. Cl. A61B 1/04; A61B 1/06

(52) U.S. Cl. 600/109; 600/163

(57) ABSTRACT

An endoscope is provided for observing an object. The endoscope includes a microfluidic device defining a well therein and a lens disposed in the well of the microfluidic device. The lens has a tunable focal length. A tuning structure is provided for tuning the focal length of the lens in response to a predetermined stimulus and an illumination fiber may be positioned adjacent to the lens for illuminating the object. An activation fiber bundle is adjacent to the tuning structure for providing the predetermined stimulus to the tuning structure. The image acquisition fiber bundle is in alignment with the lens for receiving an image therefrom.

### FIELD OF THE INVENTION

This invention relates generally to endoscopes, and in particular, to a fiber endoscope incorporating a tunable-focus microlens actuated via infrared light.

### BACKGROUND AND SUMMARY OF THE INVENTION

Optical imaging and microscopy are extremely important in biological studies and biomedical applications. As such, there has been a significant amount of research dedicated to the creation of optical components and modules at the micro-scale. This research has led to the development of such products as semiconductor-based avalanche photo-detector (APD) that can detect single photons and artificial retinas. However, compared to the maturity and tremendous success of other miniaturized systems such as integrated circuits and image processing systems, the development in miniaturized optical systems as a whole lags behind. For example, while fiber endoscopes are broadly used for diagnostics and surgery, such fiber endoscopes typically use non-tunable lenses at the distal end for imaging. Hence, operation of present fiber endoscopes requires constant and skillful manual maneuvering.

Attempts have been made to develop fiber endoscopes that utilize tunable lenses. For example, various fiber endoscopes incorporating zoom lenses have been developed. However, these fiber endoscopes utilize tiny lenses that require supporting rings to hold the bulk of the lens area. As such, these types of tunable lenses for zooming are incredibly hard to manufacture and assemble due to their small size. Other types of tunable lenses require mechanical, electrical and environmental signals for tuning. Integrating these tunable microlenses with the other optical components of the fiber endoscopes can be challenging. In addition, in the medical applications, electrical controls with high voltage or fluid circulation should be avoided.

In view of the foregoing, it can be appreciated there exists an ongoing need for fiber endoscopes incorporating tunable-focus microlenses integrated at the ends thereof that allow users to scan areas of interest with minimum movement of the endoscopes themselves. In addition, it is highly desirable to provide fiber endoscopes incorporating tunable-focus microlenses integrated at the ends thereof that allow for different depths of focus and better lateral resolution.

Therefore, it is a primary object and feature of the present invention to provide a fiber endoscope incorporating a tunable-focus microlens.

It is a further object and feature of the present invention to provide a fiber endoscope incorporating a tunable-focus microlens that allows for different depths of focus and better lateral resolution than prior fiber endoscopes.

It is a still further object and feature of the present invention to provide a fiber endoscope incorporating a tunable-focus microlens that allows a user to scan an area of interest with minimal movement of the endoscope.

It is a still further object and feature of the present invention to provide a fiber endoscope incorporating a tunable-focus microlens that is simple to utilize and easily fabricated.

In accordance with the present invention, an endoscope is provided for observing an object. The endoscope includes a microfluidic device defining a well therein and a lens disposed in the well of the microfluidic device. The lens has a tunable focal length. A tuning structure tunes the focal length of the lens in response to a predetermined stimulus. An activation fiber is positioned adjacent to the tuning structure for providing the predetermined stimulus to the tuning structure.

The tuning structure includes a hydrogel having a configuration responsive to the predetermined stimulus. The hydrogel is movable between a first configuration wherein the lens has a first focal length and a second configuration wherein the lens a second focal length in response to a predetermined stimulus. It is contemplated for the predetermined stimulus to be infrared light. An image acquisition fiber is in alignment with the lens. The image acquisition fiber receives an image from the lens. An illumination fiber is positioned adjacent to the lens for illuminating the object.

The microfluidic device may also include a plate having an aperture therethrough. The aperture communicates with the well. A first fluid is positioned on a first side of the plate and a second fluid positioned on the second side of the plate. The lens is defined by an interface of the first and second fluids. The first fluid may be an oil-based fluid and the second fluid may be a water-based fluid.

In accordance with a still further aspect of the present invention, an endoscope is provided for observing an object. The endoscope includes a microfluidic device defining a well therein. A lens is disposed in the well of the microfluidic device. The lens has a tunable focal length. A tuning structure tunes the focal length of the lens in response to a predetermined stimulus. The tuning structure includes a plurality of hydrogel posts movable between a first configuration and a second configuration for tuning the focal lengths of the plurality of lenses. An activation fiber bundle is adjacent to the tuning structure for providing the predetermined stimulus to the tuning structure. An image acquisition fiber bundle is in alignment with the lens for receiving an image therefrom.

The microfluidic device includes a plate having an aperture therethrough. The aperture communicates with the well. A plurality of hydrogel posts are received within the well of the microfluidic device. The configurations of the plurality of hydrogel posts vary in response to a predetermined stimulus. It is contemplated for the predetermined stimulus to be infrared light.

The lens includes first and second layers having an interface. The first layer is formed from an oil-based fluid and the second layer is formed from a water-based fluid. At least of a portion of the second layer of each lens is received in a corresponding well. An illumination fiber is adjacent to the lens for illuminating the object.

In accordance with a still further aspect of the present invention, an endoscope is provided for observing an object. The endoscope includes a microfluidic device having a well and a first fluid disposed in the well. A second fluid intersects the first fluid at an interface. The interface defines a lens having a focal length. A tuning structure tunes the focal length of the lens in response to a predetermined stimulus. An activation fiber bundle is adjacent to the tuning structure for providing the predetermined stimulus to the tuning structure.

The microfluidic device includes a plate having an aperture therethrough. The aperture communicates with the well. The tuning structure includes a plurality of hydrogel posts received in the well. Each hydrogel post is movable between a first configuration and a second configuration for tuning the focal lengths of the plurality of lenses. The configurations of the plurality of hydrogel posts vary in response to a predetermined stimulus. The plurality of hydrogel posts include water-soluble gold nanoparticles therein. The gold nanoparticles optically absorb infrared light. Each fiber of an image acquisition fiber bundle is in alignment with the lens for receiving an image therefrom.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is a schematic view of an endoscope in accordance with the present invention;

FIG. 2 is a cross-sectional view showing a first step in the fabrication of a micolens for the endoscope of FIG. 1;

FIG. 3 is a cross-sectional view showing a second step in the fabrication of the micolens for the endoscope of FIG. 1;

FIG. 4 is a cross-sectional view showing a third step in the fabrication of the micolens for the endoscope of FIG. 1;

FIG. 5 is a cross-sectional view showing a fourth step in the fabrication of the micolens for the endoscope of FIG. 1;

FIG. 6 is a cross-sectional view of a fifth step in the fabrication of the micolens for the endoscope of FIG. 1;

FIG. 7 is a cross-sectional view of a sixth step in the fabrication of the micolens for the endoscope of FIG. 1;

FIG. 8 is a cross-sectional view of a seventh step in the fabrication of the micolens for the endoscope of FIG. 1;

FIG. 9 is a cross-sectional view of an eighth step in the fabrication of the micolens for the endoscope of FIG. 1;

FIG. 10 is a cross-sectional view showing the micolens for the endoscope of the present invention in a first configuration;

FIG. 11 is a cross-sectional view showing the micolens for the endoscope of the present invention in a second configuration; and

FIG. 12 is a schematic view of an alternate embodiment of an endoscope in accordance with the present invention.

### DETAILED DESCRIPTION OF THE DRAWINGS

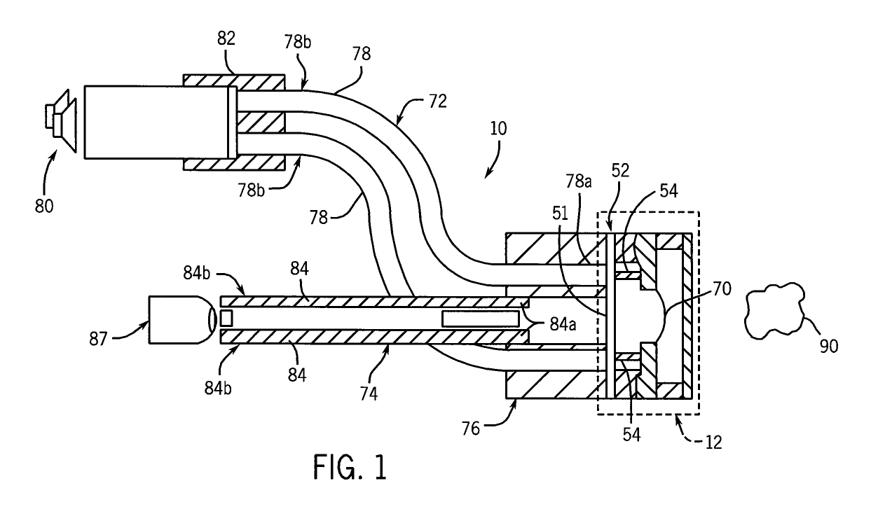

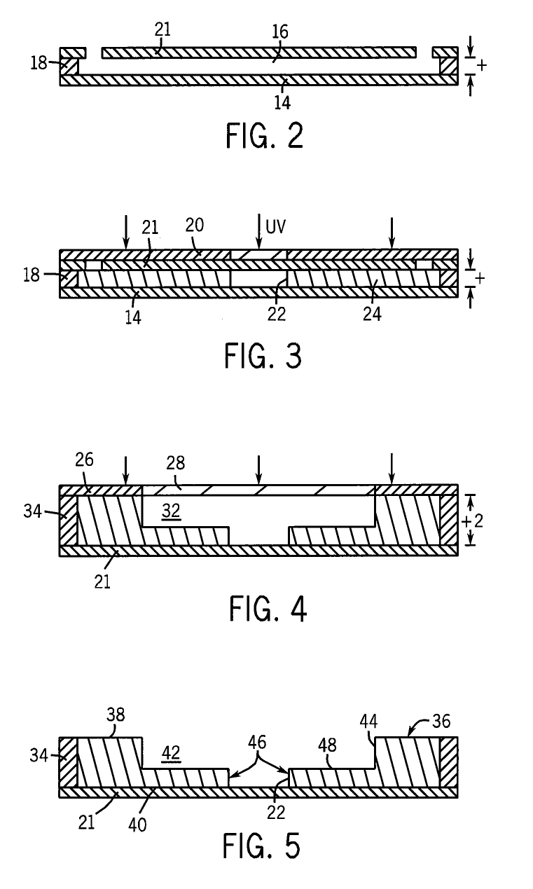

Referring to FIG. 1, a schematic view of a fiber endoscope in accordance with the present invention is generally designated by reference numeral 10. Fiber endoscope 10 includes a tunable-focus microlens 12. Referring to FIGS. 2-9, in order to fabricate microlens 12 of fiber endoscope 10, cartridge 21 is fixed on substrate 14 so as to define cavity 16 therebetween. Cartridge 21 is spaced from substrate 14 by spacer 18 such that cavity 16 has a desired thickness t, e.g. 250 micrometers (μm). Cavity 16 is filled with a liquid photopolymer and first photomask 20, corresponding in size and shape to a desired aperture 22, is positioned between cartridge 21 and an ultraviolet light source, FIG. 3. Ultraviolet light is directed toward the liquid photopolymer such that a portion of the liquid photopolymer exposed to the ultraviolet light solidifies and forms first layer 24. Thereafter, the portion of the photopolymer that remains in a liquid state is flushed from the cartridge leaving aperture 22 within first layer 24.

After fabrication of aperture 22 in first layer 24, substrate 14 and spacer 18 are peeled off first layer 24 and first layer 24 is flipped over. Second cartridge 26 having second photomask 28 incorporated therein is positioned above first layer 24 so as to define cavity 32 therebetween, FIG. 4. Second cartridge 26 is spaced from first layer 24 by spacer 34 such that cavity 32 has a desired thickness t2, e.g. 800 μm. Cavity 32 is filled with a liquid photopolymer and ultraviolet light is directed toward the liquid photopolymer such that a portion of the liquid photopolymer exposed to the ultraviolet light solidifies and bonds to first layer 24 so as to form plate 36. Thereafter, the portion of the photopolymer that remains in a liquid state is flushed from plate 36.

Plate 36 is defined by first and second spaced surfaces 38 and 40, respectively. Well or cavity 42 in plate 36 is defined by side wall 44 projecting vertically from first surface 38 and aperture 22 in plate 36 is defined by side wall 46 projecting vertically from second surface 40. Side walls 44 and 46 of plate are interconnected by horizontal surface 48. Side walls 44 and 46 and surface 48 of plate 36 are rendered with a plasma treatment from hydrophobic to hydrophilic, FIG. 5, for reasons hereinafter described.

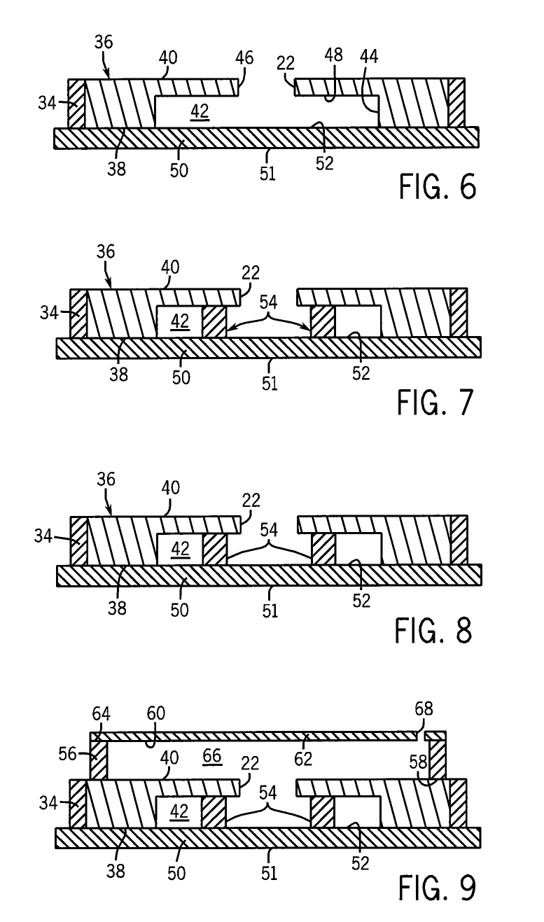

After treatment of plate 36 with plasma, cartridge 21 is removed from second surface 40 thereof and plate 36 is flipped over, FIG. 6. Thereafter, first surface 38 of plate 36 is bonded onto upper surface 50 of glass slide 52 and cavity 42 in plate is filled with an infrared-light-responsive hydrogel pre-polymer solution having gold nanoparticles entrapped therein. The gold nanoparticles in the infrared-light-responsive hydrogel pre-polymer solution have high absorption of light in the infrared range. A third photomask (not shown) having a plurality of apertures therein is positioned between plate 36 and the ultraviolet light source. The apertures in the third photomask correspond in size and shape to a plurality of hydrogel posts 54 to be formed in cavity 42 at locations circumferentially spaced about aperture 22 in plate 36. Ultraviolet light is directed toward the infrared-light-responsive hydrogel pre-polymer solution such that a portion of the infrared-light-responsive hydrogel pre-polymer solution is exposed to the ultraviolet light through the apertures in the third photomask and solidifies so as to form the plurality of hydrogel posts 54 in cavity 42 in plate 36, FIG. 7. Thereafter, the portion of the infrared-light-responsive hydrogel pre-polymer solution that remains in a liquid state is flushed from cavity in plate 36. It can be appreciated that infrared-light-responsive hydrogel posts 54 may have other configurations, (e.g., a single ring instead of the plurality of hydrogel posts) without deviating from the scope of the present invention. Second surface 40 of plate 36 is treated to achieve better hydrophobicity, e.g. bushing an octadecyltrichlorosilane (OTS) solution diluted by hexadecane (0.2% v/v) thereon, FIG. 8.

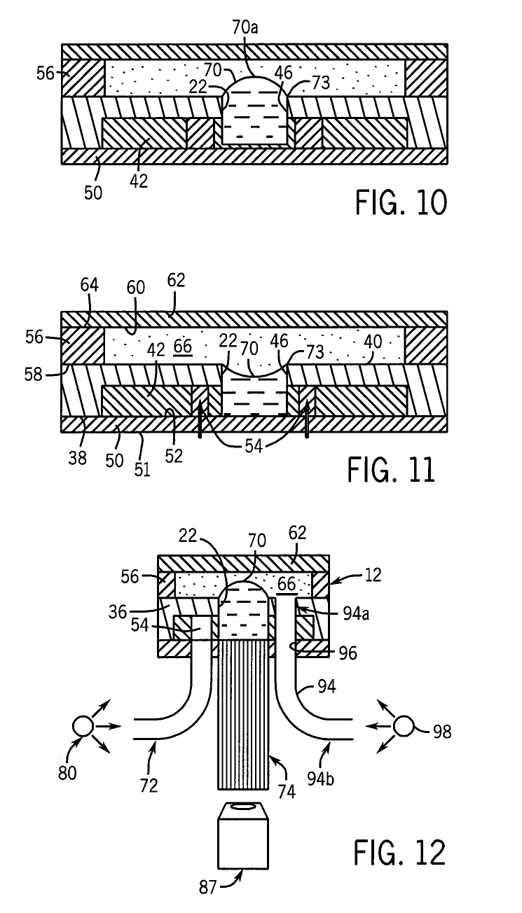

Finally, lower surface 58 of polydimethylsiloxane (PDMS) ring 56 that has been treated with plasma to improve its adhesion is bonded to second surface 40 of plate 36 adjacent to the outer periphery thereof. Lower surface 60 of glass slide 62 is bonded to upper surface 64 of ring 56 so as to form chamber 66 between second surface 40 of plate 36 and lower surface 60 of glass slide 62, FIG. 9. Glass slide 62 includes aperture 68 therethrough that communicates with chamber 66 so as to allow a user to fill cavity 42 and chamber 66. More specifically, water is injected into cavity 42 of plate 36 through aperture 68 in glass slide 62 and aperture 22. In addition, oil is injected into chamber 66 through aperture 68 in glass slide 62 such that water-oil interface 70 defining a liquid meniscus generally designated by the reference numeral 70a is formed at the hydrophobic-hydrophilic contact line 73, namely, the intersection of side wall 46 and second surface 40 of plate 36, FIGS. 10-11. It can be appreciated that the oil in chamber 66 prevents the evaporation of the water in cavity 42. In addition, the water-oil meniscus forms a lens since the refractive index of oil (1.48) is higher than that of water (1.33). Other types of lenses are, for example, disclosed in commonly owned U.S. patent application Ser. No. 11/442,927, filed on May 30, 2006, entitled “Variable-Focus Lens Assembly, and incorporated by reference herein in its entirety. Thereafter, aperture 68 in glass slide 62 is sealed in any conventional manner.

With microlens 12 assembled, it is noted that oil-water interface 70 is pinned at the edge of aperture 22 as a result of side wall 46 of plate 36 being hydrophilic and second surface 40 of plate 36 being hydrophobic. Consequently, hydrophobic-hydrophilic contact lines are formed that pin oil-water interface 70 via surface tension. The stationary pinned contact line translates a change in the water volume in cavity 42 into a change in the contact angle of the water-oil interface 70, and thus, the focal length of the lens. Contact angle θ of water-oil interface 70 may attain any value within a certain range by varying the pressure difference P across water-oil interface 70.

In addition, it is noted that when hydrogrel posts 54 are exposed to a predetermined stimulus (e.g. infrared light), hydrogel posts 54 expand or contract by absorbing and releasing water, respectively, provided in cavity 42 via their hydrogel network interstitials. The expansion and contraction of hydrogel posts 54 is depicted in phantom in FIGS. 10-11. This, in turn, results in a volume change in the water deposited in 42 in plate 36. The net physical volume change in both the hydrogel posts 54 and the water received in cavity 42 causes a change in the pressure difference across the water-oil interface 70 which directly determines the shape thereof. More specifically, hydrogel posts 54 contract in response exposure to infrared light incident onto them, pulling down oil-water interface 70 towards the water and eventually into the water, rendering a more convergent lens, FIG. 11. When the infrared light is removed, hydrogel microposts 54 expand, thereby pushing oil-water interface 70 back towards the oil, thus restoring a less convergent lens, FIG. 10.

Referring back to FIG. 1, in order to assemble fiber endoscope 10, first and second sets of optical fibers 72 and 74, respectively, are bound to lower surface 51 of glass slide 52 of microlens 12 by machined adapter 76. More specifically, first ends 78a of optical fibers 78 of first set of optical fibers 72 are aligned with corresponding hydrogel posts 54 for actuating the hydrogel posts 54, as heretofore described. Second ends 78b of optical fibers 78 of first set of optical fibers 72 are interconnected to infrared light source 80 by adapter 82. First ends 84a of optical fibers 84 of second set of optical fibers 74 are aligned with water-oil interface 70 for image acquisition. Second ends 84b of optical fibers 84 of second set of optical fibers 74 are interconnected to charge coupled device (CCD) camera 87 in a conventional manner.

In operation, fiber endoscope 10 is located within a body in a conventional manner to view a desired object 90. Thereafter, infrared light from infrared light source 80 is transmitted via optical fibers 78 of first set of optical fibers 72 to hydrogel posts 54 so as to cause hydrodel posts 54 to contract in response thereto, FIG. 10. As hydrogel posts 54 contract, oil-water interface 70 is pulled down towards the water thereby varying the focal length of the lens defined by water-oil interface 70. Alternatively, once the infrared light from infrared light source 80 is terminated, hydrodel posts 54 expand in response thereto, FIG. 11. As hydrogel posts 54 expand, oil-water interface 70 is pushed back towards the oil thereby, once again, varying the focal length of the lens defined by water-oil interface 70. It can be appreciated that by controlling the transmission of infrared light supplied to the microlens, a user may tune the focal length of the lens to a user desired location. Images from the tunable-focus lens defined by water-oil interface 70 of object 90 are transferred to CCD camera 87 via second set of optical fibers 74.

It can be appreciated that the aforementioned lens formed by oil-water interface 70 can focus on objects at different distances. By causing the hydrogel posts 54 to change their volumes, the lens can be tuned to focus on desired targets. Due to a hydrogel's ability to convert chemical energy to mechanical energy, hydrogel posts 54 simultaneously exhibit both sensing and actuating functions to respond to local environments.

Referring to FIG. 12, it is contemplated to illuminate object 70 to facilitate image acquisition. More specifically, it is contemplated for plate 36 to include second aperture 92 therein which is adapted from receiving a first end 94a of illumination optical fiber 94. Illumination optical fiber 94 extends through aperture 96 in glass slide 52 and has a second end 94b coupled to illumination light source 98. In operation, light from light source 98 is transmitted via illumination optical fiber 94 to the first end 94a thereof. Light is dispersed from first end 94a of illumination optical fiber 94 so as to illuminate object 90.

Various modes of carrying out the invention are contemplated as being within the scope of the following claims particularly pointing out and distinctly claiming the subject matter, which is regarded as the invention.

We claim:

1. An endoscope for observing an object, comprising:
a microfluidic device defining a well therein;
a lens disposed in the well of the microfluidic device, the lens having a tunable focal length;
a tuning structure for tuning the focal length of the lens in response to a predetermined stimulus;
an activation fiber adjacent to the tuning structure for providing the predetermined stimulus to the tuning structure.

2. The endoscope of claim 1 wherein the tuning structure includes a hydrogel, the hydrogel having a configuration responsive to the predetermined stimulus.

3. The endoscope of claim 2 wherein the hydrogel is movable between a first configuration wherein the lens has a first focal length and a second configuration wherein the lens has a second focal length in response to a predetermined stimulus.

4. The endoscope of claim 3 wherein the predetermined stimulus is infrared light.

5. The endoscope of claim 1 further comprising an image acquisition fiber in alignment with the lens, the image acquisition fiber receiving an image from the lens.

6. The endoscope of claim 1 wherein the microfluidic device includes a plate having an aperture therethrough, the aperture communicating with the well.

7. The endoscope of claim 6 further comprising first and second fluids, the first fluid positioned on a first side of the plate and the second fluid positioned on the second side of the plate.

8. The endoscope of claim 7 wherein the lens is defined by an interface of the first and second fluids.

9. The endoscope of claim 7 wherein the first fluid is an oil-based fluid and the second fluid is a water-based fluid.

10. The endoscope of claim 1 further comprising an illumination fiber adjacent to the lens for illuminating the object.

11. An endoscope for observing an object, comprising:
a microfluidic device defining a well therein;
a lens disposed in the well of the microfluidic device, the lens having a tunable focal length;
a tuning structure for tuning the focal length of the lens in response to a predetermined stimulus, the tuning structure including a plurality of hydrogel posts movable between a first configuration and a second configuration for tuning the focal lengths of the plurality of lenses;
an activation fiber bundle adjacent to the tuning structure for providing the predetermined stimulus to the tuning structure; and
an image acquisition fiber bundle in alignment with the lens for receiving an image therefrom.

12. The endoscope of claim 11 further comprising an illumination fiber adjacent to the lens for illuminating the object.

13. The endoscope of claim 11 wherein the microfluidic device includes a plate having an aperture therethrough, the aperture communicating with the well.

14. The endoscope of claim 11 wherein the plurality of hydrogel posts are received within the well of the microfluidic device.

15. The endoscope of claim 11 wherein the configurations of the plurality of hydrogel posts vary in response to a predetermined stimulus.

16. The endoscope of claim 15 wherein the predetermined stimulus is infrared light.

17. The endoscope of claim 11 wherein the lens includes first and second layers having an interface.

18. The endoscope of claim 17 wherein the first layer is formed from an oil-based fluid and the second layer is formed from a water-based fluid.

19. The endoscope of claim 17 wherein at least of a portion of the second layer of each lens is received in a corresponding well.

20. An endoscope for observing an object, comprising:
a microfluidic device including a well;
a first fluid disposed in the well;
a second fluid intersecting the first fluid at an interface, the interface defining a lens having a focal length;
a tuning structure for tuning the focal length of the lens in response to a predetermined stimulus; and
an activation fiber bundle adjacent to the tuning structure for providing the predetermined stimulus to the tuning structure.

21. The endoscope of claim 20 wherein the microfluidic device includes a plate having an aperture therethrough, the aperture communicating with the well.

22. The endoscope of claim 20 wherein the tuning structure includes a plurality of hydrogel posts engageable received in the well, each hydrogel post movable between a first configuration and a second configuration for tuning the focal lengths of the plurality of lenses.

23. The endoscope of claim 22 wherein the configurations of the plurality of hydrogel posts vary in response to a predetermined stimulus.

24. The endoscope of claim 22 wherein the plurality of hydrogel posts include water-soluble gold nanoparticles therein, the gold nanoparticles optically absorbing infrared light.

25. The endoscope of claim 20 further comprising an image acquisition fiber bundle, each fiber of the image acquisition fiber bundle in alignment with the lens to receive an image therefrom.
